(19) United States
(12) Patent Application Publication
(10) Pub. No.: US 2026/0115380 A1
(43) Pub. Date: Apr. 30, 2026

(54) USER-WEARABLE INFUSION PUMP SYSTEM WITH ANGLED CANNULA

(71) Applicant: Tandem Diabetes Care, Inc., San Diego, CA (US)

(72) Inventors: Justin Coker, Roseville, CA (US); Kennie Hsu, Tustin, CA (US); Amrith Karunaratne, Irvine, CA (US)

(21) Appl. No.: 19/003,164

(22) Filed: Dec. 27, 2024

Related U.S. Application Data
(60) Provisional application No. 63/615,919, filed on Dec. 29, 2023.

(51) Int. Cl. A61M 5/142; A61M 5/158; A61M 39/02

(52) U.S. Cl. CPC A61M 5/14244; A61M 5/158; A61M 39/0208

(57) ABSTRACT

Embodiments of the present disclosure relate to an infusion pump system including a holder or carrier for a user-wearable infusion pump that can be worn on the patient's body. Embodiments depicted and described herein provide a more versatile, secure, and user-friendly way to hold and carry a user-wearable infusion pump, including providing a more versatile way for interfacing a cannula inserted into the user's skin to deliver medicament to the user from the pump with the pump system.

### PRIORITY CLAIM

The present application claims the benefit of U.S. Provisional Application No. 63/615,919 filed Dec. 29, 2023, which is hereby incorporated by reference herein.

### TECHNICAL FIELD

The present disclosure relates to medical pumps for delivering medicament to a patient and, more specifically, to a user-wearable pump.

### BACKGROUND

There are many applications in academic, industrial, and medical fields that benefit from devices and methods that are capable of accurately and controllably delivering fluids, such as liquids and gases, that have a beneficial effect when administered in known and controlled quantities. Such devices and methods can be particularly useful in the medical field where treatments for many patients include the administration of a known amount of a substance at predetermined intervals.

One category of devices for delivering such fluids is that of pumps that have been developed for the administration of insulin and other medicaments for those suffering from both type I and type II diabetes. Some pumps configured as portable infusion devices can provide continuous subcutaneous medicament injection and/or infusion therapy for the treatment of diabetes. Such therapy may include, e.g., the regular and/or continuous injection or infusion of insulin into a person suffering from diabetes and offer an alternative to multiple daily injections of insulin by an insulin syringe or an insulin pen. Such pumps can be ambulatory/portable infusion pumps that are worn by the user and may use replaceable cartridges. Such pumps can deliver medicaments other than or in addition to insulin, such as glucagon. Examples of such pumps and various features that can be associated with such pumps include those disclosed in U.S. Pat. No. 8,287,495, which is hereby incorporated herein by reference in its entirety.

One type of pump that has been developed is a patch pump, or micro pump. Patch pumps generally are small pumps that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Not unlike other types of pumps, but perhaps more typically, patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament. Still other patch pumps may include a rechargeable durable component as well as a refillable and reusable medicament cartridge.

### SUMMARY

Embodiments of the present disclosure relate to an infusion pump system including a holder or carrier for a user-wearable infusion pump that can be worn on the patient's body. Embodiments depicted and described herein provide a more versatile, secure, and user-friendly way to hold and carry a user-wearable infusion pump, including providing a more versatile way for interfacing a cannula inserted into the user's skin to deliver medicament to the user from the pump with the pump system.

In an embodiment, a user-wearable infusion pump system can include a user-wearable infusion pump configured to contain a medicament and a pump holder configured to releasably hold the user-wearable infusion pump. A cannula can be configured to be inserted through the tray and into a body of a user to deliver medicament from the user-wearable infusion pump into the body of the user. The cannula can be configured to be inserted into the body of the user at an angle between parallel and perpendicular to the base of the pump holder. The user-wearable infusion pump is configured to be slid generally parallel along the base of the pump holder to releasably lock the user-wearable infusion pump onto the pump holder and interface the user-wearable infusion pump with the cannula.

In an embodiment, user-wearable infusion pump system can include a user-wearable infusion pump configured to contain a medicament and a pump holder configured to releasably hold the user-wearable infusion pump. The pump holder can include a base on which the user-wearable infusion pump is configured to retained. A cannula can be configured to be inserted through the pump holder and into a body of a user to deliver medicament from the user-wearable infusion pump into the body of the user. The cannula can be configured to be inserted into the body of the user at an angle between parallel and perpendicular to the base of the pump holder. The user-wearable infusion pump can be configured to be slid generally parallel along the base of the pump holder to releasably lock the user-wearable infusion pump onto the pump holder and interface the user-wearable infusion pump with the cannula.

In embodiments, a pump holder for user-wearable infusion pump system is configured to releasably retain a user-wearable infusion pump on a body of a user. The pump holder can include a base on which the user-wearable infusion pump is configured to be retained and a cannula port including a cannula opening configured to receive a cannula inserted into the body of the user. The cannula opening can be oriented at an angle between parallel and perpendicular to the base. The user-wearable infusion pump can be configured to be slid generally parallel along the base to releasably lock the user-wearable infusion pump onto the pump holder and interface the user-wearable infusion pump with the cannula.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict a patch pump system according to an embodiment of the disclosure.

FIGS. 2A-2B depict a patch pump system according to an embodiment of the disclosure.

FIG. 3 depicts a patch pump system according to an embodiment of the disclosure.

FIG. 4 depicts a patch pump system according to an embodiment of the disclosure.

FIGS. 5A-5B depict a patch pump system according to an embodiment of the disclosure.

FIGS. 6A-6B depict the patch pump system of FIGS. 5A-5B.

FIGS. 7A-7C depict a patch pump system according to an embodiment of the disclosure.

FIGS. 8A-8B depict the patch pump system of FIGS. 7A-7C.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

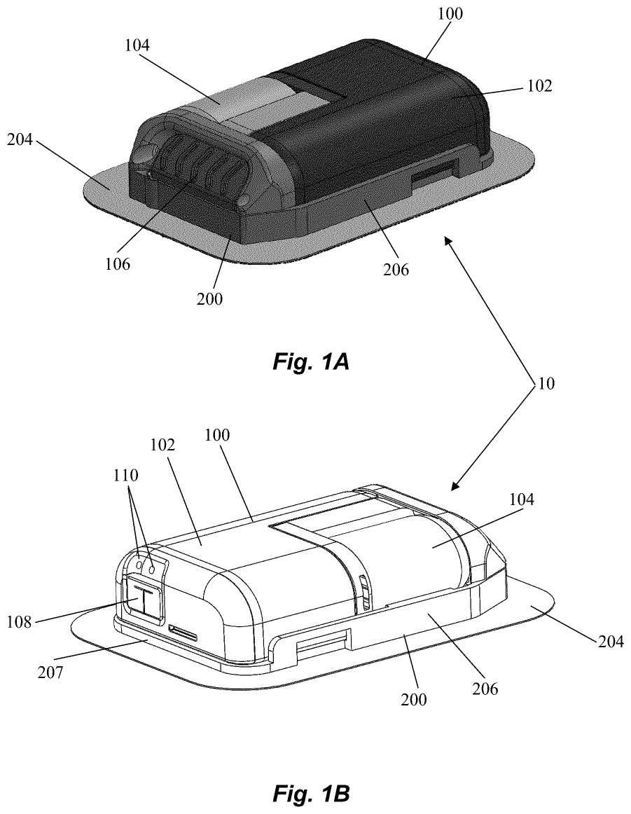

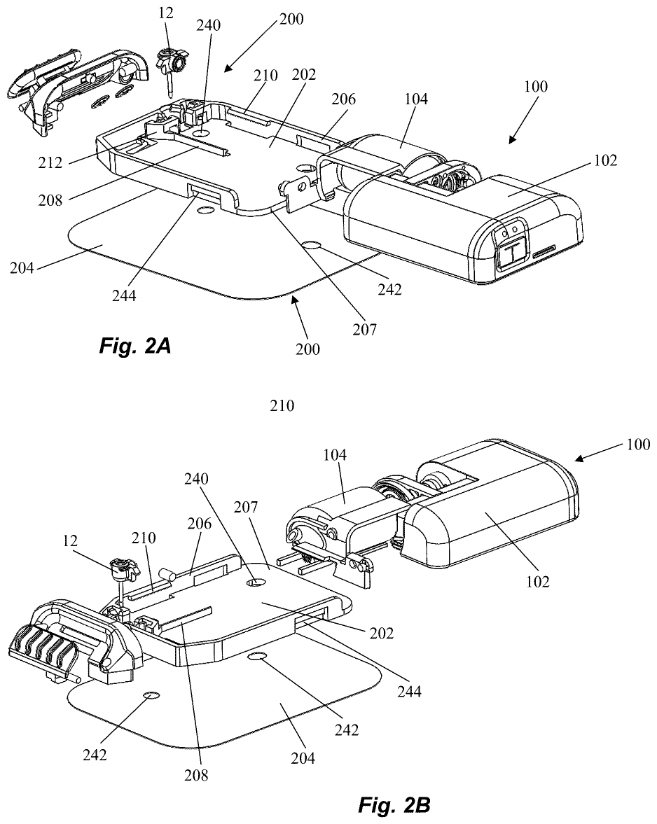

FIGS. 1A-1B and 2A-2B depict an ambulatory infusion pump system 10 according to an embodiment of the disclosure. System 10 can include an infusion pump 100 and a pump holder or tray 200 that can be used to releasably contain infusion pump 100 and that can be worn on a body of a user.

Pump 100 can include a drive unit 102 including a drive mechanism that mates with a recess in a medicament cartridge 104 to releasably hold the cartridge 104 on the drive unit 102. Further details regarding embodiments of a drive mechanism for delivering a medicament such as insulin from the medicament cartridge to a user and for attaching the cartridge to the drive unit can be found in U.S. Pat. Nos. 10,279,107 and 9,993,595, each of which is incorporated herein by reference in its entirety. In some embodiments, drive unit 102 is a durable component having a rechargeable battery and cartridge 104 is disposable and intended to be disposed of after the medicament in cartridge has been used. In other embodiments, cartridge can be refillable such that both components are durable. Pump 102 can further include a switch 106 configured to releasably lock the pump 102 onto the pump holder 200.

In embodiments, pump 100 can interface with a cannula 12 that extends through the pump holder 200 to deliver medicament to an infusion site on the user's body directly beneath the pump holder 200. In other embodiments, pump 100 can interface with infusion tubing to deliver medicament from the pump through the tubing to an infusion site displaced from the pump holder 200. In some embodiments, the pump 100 can be configured to interchangeably be used with a cannula directly beneath the pump holder 200 or an infusion site displaced from the holder 200 via infusion tubing. In some of those embodiments, drive unit 102 can be configured to attach to alternative cartridge types, with a first cartridge type including infusion tubing and a second cartridge type configured to deliver through a cannula extending directly beneath the pump 100. In various embodiments, pump 100 can be disconnected from a user's body and reattached using the same infusion site to enable the user to remove the pump for a limited period of time for certain activities, such as, e.g., charging the battery, taking a shower, playing sports, etc. without having to insert a cannula at a new infusion site.

In embodiments, pump includes a processor that controls the operations of the pump and may communicate in either one-way or two-way modes to, e.g., receive operational commands and/or other signals, including data, from a separate device and/or, e.g., to send signals, including data to a separate device. Pump can include one or more buttons configured to cause the processor to initiate one or more functions. In the depicted embodiment, pump 100 includes only a single button 108, although more than one button may be present on pump 100. Button 108 can be configured to, for example, initiate delivery of medicament (e.g., a “quick bolus”). Any single button such as button 108 can be utilized to execute a plurality of functions or operations. For example, a single press of button may initiate one function, holding the button down for a predetermined period of time may initiate another function, etc. Because the depicted pump 100 optionally does not itself include a display or user interface, information and feedback regarding medicament delivery or dosing or other functions initiated with button 108 can be communicated to and displayed on a remote control device or other device having a display and/or other type of user interface.

In embodiments, pump 100 includes a light source, such as a light emitting diode (LED) 110. Light source 110 can be configured to provide user feedback regarding user input and/or the performance of a desired function. For example, in one embodiment, light source 110 can illuminate or blink one or more times to indicate that the one or more buttons 108 have been activated and/or that a desired function has been initiated. In one embodiment, pump 100 can additionally and/or alternatively vibrate and/or provide audible notifications to indicate that the one or more buttons 108 have been activated and/or that a desired function has been initiated or, e.g., to provide user feedback regarding user input and/or the performance of the desired function. Illumination of light source 110 and/or vibrations and/or audible notifications may be executed in any number of patterns, frequencies, durations, sequences, combinations, colors, brightness levels, etc. to indicate particular information, such as particular input received and/or particular functions or operations enabled and/or initiated, to the pump user or caregiver. In the depicted embodiment, the pump 100 includes two indicator lights 110.

Pump holder 200 can include a tray body 202 having a shape generally matching the shape of pump 100 and an adhesive patch 204 configured to releasably retain the pump holder 200 and pump 100 on the user's body. One or more perimeter walls 206 can extend around at least a portion of the perimeter of the tray body 202 to aid in retaining and guiding the pump 100 onto the holder 200. In the depicted embodiment, tray body 202 can include one end 207 that does not include a perimeter wall to aid in sliding pump 102 onto tray body 202. Pump holder 200 can include various other features that aid in retaining and/or guiding the pump onto holder 200, including a guide rail 208, retention projection 210 and a locking ramp 212. Pump holder 200 and patch 204 can include aligned apertures 240, 242 and retention walls 206 can include slots 244 that aid in temporarily retaining pump holder 200 in and releasing pump holder 200 from an insertion or applicator device configured to apply the pump holder 200 onto the user's skin.

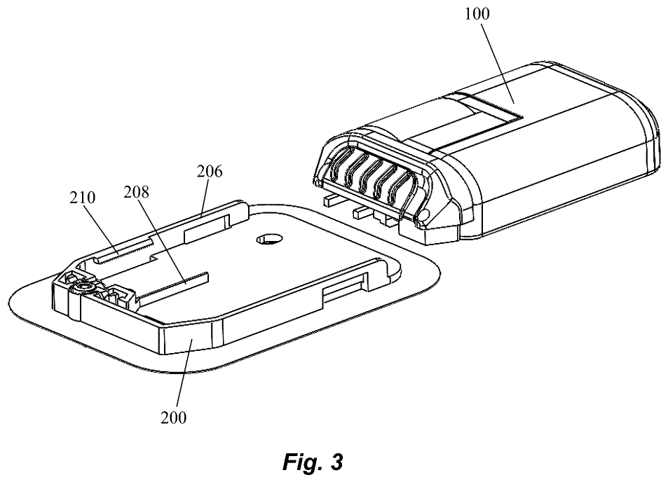

Referring to FIG. 3, pump 102 is designed to be inserted onto holder 200 by positioning pump adjacent the open end 207 and laterally sliding pump 100 along tray body 202. The interaction of lower guide rail 128 of pump sliding along guide rail 208 of holder 200 and wall guide rail 126 of pump along perimeter wall 126 and beneath retention projection 210 of holder ensures proper alignment of the pump 100 onto the holder 200.

As noted above, pump 100 can interface with a cannula 12 that extends through the tray and into the body of the user for delivery of medicament from the pump 100 into the user. In the embodiment depicted above, the cannula 12 extends generally perpendicularly through the tray. However, some cannula technology is configured to be inserted at an angle into the body, which can increase patient comfort and reduce inflammation. Such technology is disclosed in U.S. Patent Publication Nos. 2021/0402084 and 2022/0226568, each of which is hereby incorporated by reference herein in its entirety. Use of such a cannula can enable extended wear of the pump of 7 days or more at a single location (most infusion sets are designed for only 3 days of wear because the infusion site needs to be rotated).

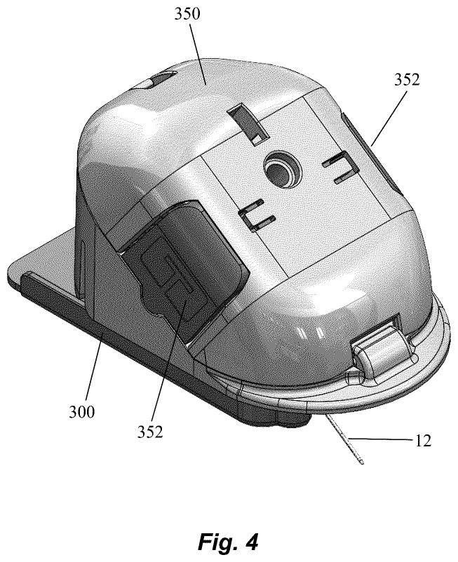

Disclosed herein are embodiments that provide the advantages of an angled cannula while maintaining the simplicity and user convenience of horizontally sliding the pump onto the tray to interface the pump with the cannula. Referring to FIG. 4, an angled cannula insertion device 350 can be employed to insert a cannula 12 at an angle through a pump holder 300. One example insertion device 350 is disclosed in U.S. Patent Publication No. 2021/0402084, previously incorporated by reference herein. Insertion device 350 can include features that mechanically interface with features on the holder 300 to ensure proper alignment for insertion of cannula 12 through a cannula port on the pump holder, described in more detail below. In some embodiments, insertion device 350 can further includes features to releasably retain pump holder 300 for positioning of holder on the user's body. In such an embodiment, actuation of one or more buttons 352 on insertion device may release the pump holder 300 from the insertion device 350 and insert the cannula 12 into the user's body with a single actuation of the one or more buttons 352.

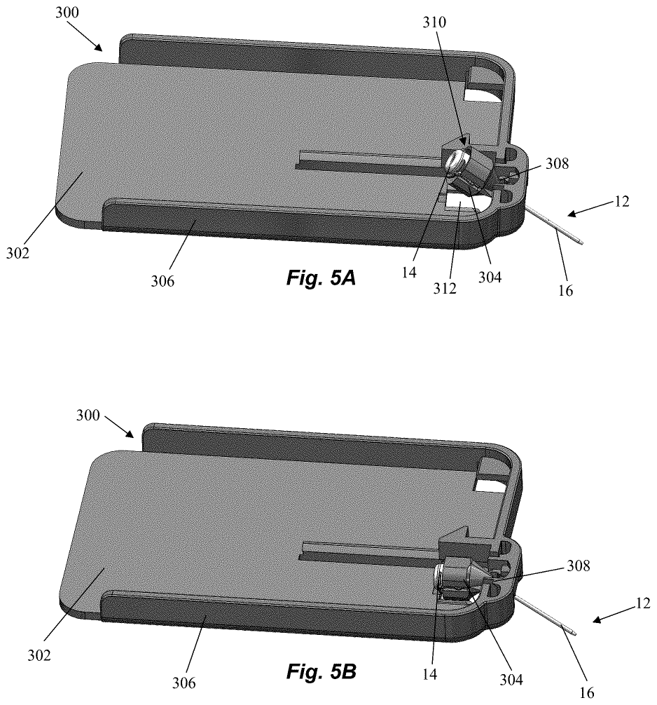

Referring now to FIGS. 5A-5B, pump holder 300 can include a tray body 302 including similar alignment and pump retention features to those of pump holder 200. Tray body 302 can further include a cannula port 304 connected to a retention wall 306 of tray body 302 with a living hinge 308. In some embodiments, such as those in which the tray body 302 is formed of a thermoplastic such as polypropylene, the cannula port 304 and living hinge 308 can be molded as part of a unitary construct with the tray. In other embodiments, the cannula port 304 and living hinge 308 can be formed as a separate component that is attached to the tray by, for example, heat staking, ultrasonic welding, etc.

As shown in FIG. 5A, the cannula port 304 is initially in a raised position, via living hinge 208, relative to the retention wall 306 with a cannula opening 310 of the cannula port angled relative to a base of tray body 302. This facilitates the angled insertion of an elongate flexible delivery shaft 16 of the cannula 12 into the cannula opening 310 and through the tray body 302 into the user's skin with the angled cannula insertion device 350. A barrel 14 of the cannula 12 is seated in the cannula opening 310. Following this initial insertion step, the living hinge 308 is pivoted downwardly to align the cannula opening 310, and therefore the barrel 14 of the cannula 12, perpendicularly to the tray body 302. In some embodiments, this cannula port 304 pivoting step is done automatically by the insertion device 350. While this step causes the cannula delivery shaft 16 to bend, embodiments can include anti-kinking features, such as, for example, an internal coil, that enables the delivery shaft 16 to bend without inhibiting flow of medicament through shaft 16. Other anti-kinking features can additionally or alternatively be employed, such as a ribbed and/or fluted inner lumen or a rigid polymer coextruded into the cannula. One example of a “fluted” cannula is disclosed in U.S. Patent Publication No. 2023/0277765, which is hereby incorporated herein by reference.

In some embodiments, cannula port 304 pivots down to interface with an opening 312 through tray body 302. Opening 312 can ensure proper alignment of cannula barrel 14 for interfacing with a pump by being sized to receive a portion of cannula port 304 therein while preventing cannula port 304 from pivoting further through tray body 302. As can be seen in comparing FIGS. 5A and 5B, delivery shaft 16 of cannula remains at the same angle relative to the tray 302 within the user's skin when the cannula port 304 is pivoted downwardly to the second position depicted in FIG. 5B. In some embodiments, the insertion device 350 automatically causes the cannula port 304 to pivot as part of the cannula insertion process carried out by insertion device 350.

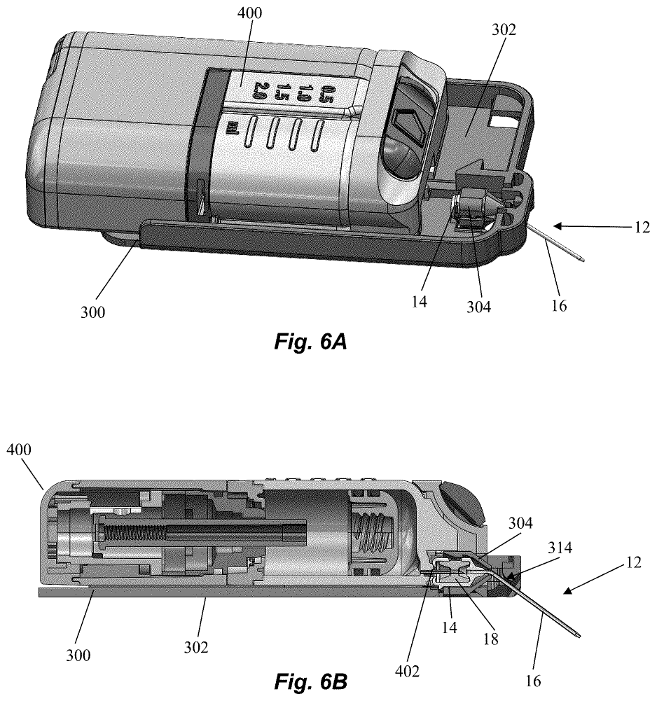

With the cannula opening 310 and barrel 14 aligned perpendicularly with the tray body 302 as shown in FIG. 5B, a pump 400 configured to interface with an angled cannula can be slid horizontally along, i.e., parallel with, tray body 302 to releasably attach pump 100 to pump holder 300. As shown in FIG. 6B, a transfer needle 402 on pump 400 can be positioned to be inserted through a septum 18 in the barrel 14 of the cannula 12 positioned within the cannula opening 310 of the cannula port 304 to fluidly connect the cannula 12 with the pump 400. As also shown in this figure, flexible delivery shaft 16 is bent at cannula port 304 to enable the opening of barrel 14 to be perpendicular to the tray body 302 with the delivery shaft 16 at an angle to the tray body 302 where it extends out of a cannula slot 314 in the the holder 300 and into the user's body. In this manner, the embodiment of FIGS. 4-6B provides the convenience of any easy attachment of the pump to a pump holder with the advantages associated with a cannula that is angled relative to the pump and the tray.

Use of a cannula port that can pivot between a first position for insertion of the cannula at an angle into the skin and a second position to interface with the pump enables the device to employ only a single septum, which simplifies the design of the fluid path, reduces the internal volume needed in the cannula port and reduces potential leak paths. In other embodiments, the cannula can be inserted at a 90 degree angle through the tray (i.e., perpendicularly to the tray) and can utilized two septa (i.e., one through which the cannula is inserted and another that interfaces with the transfer needle 402 of the pump) or an “H” shaped septum as disclosed in the publications incorporated by references above.

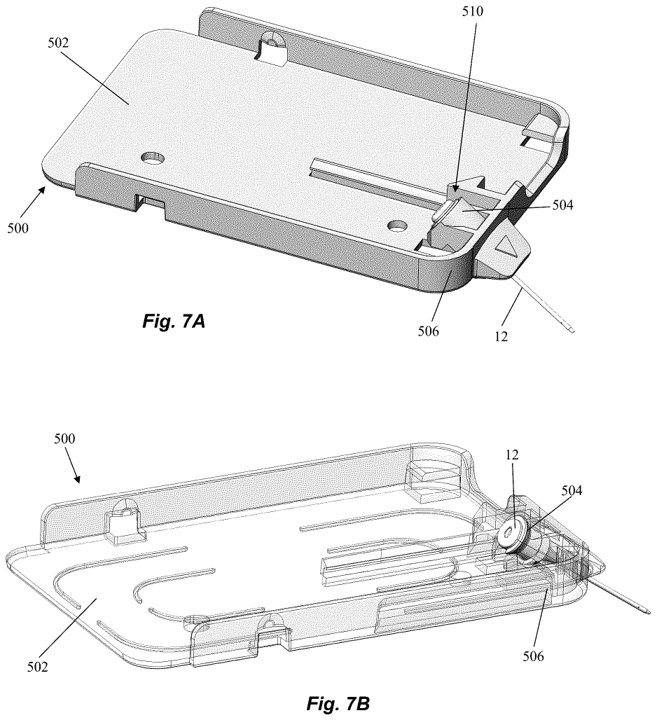

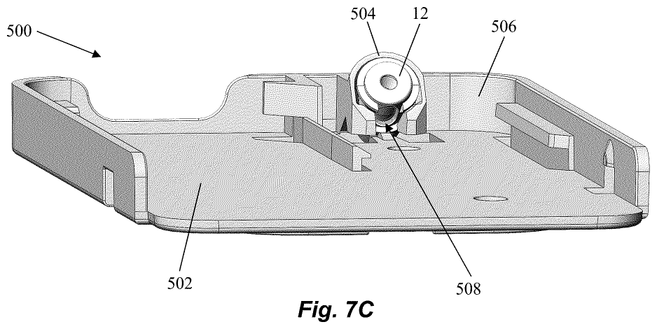

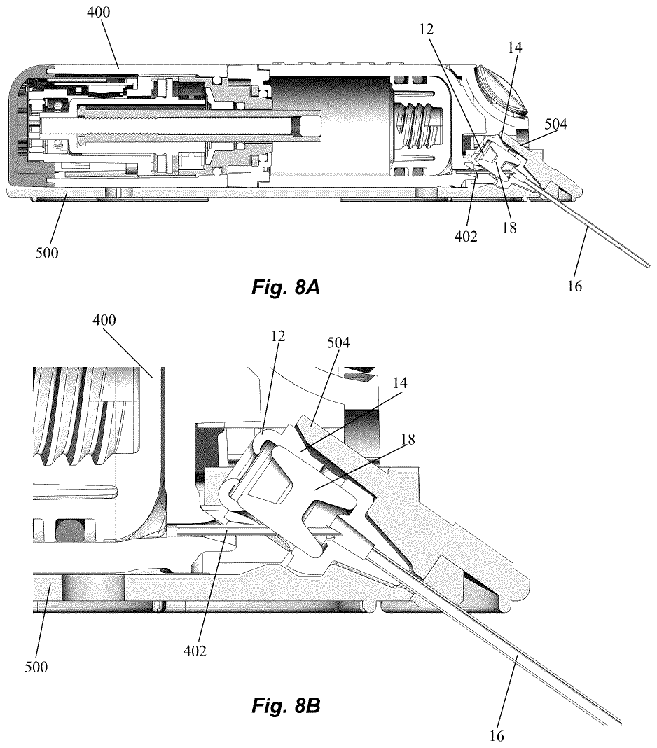

Referring now to FIGS. 7A-7C and 8A-8B, a pump holder 500 according to another embodiment can include a tray body 502 including similar alignment and pump retention features to those of pump holder 200 and pump holder 300. Tray body 502 can further include a cannula port 504 connected to a retention wall 506 of tray body 502.

Unlike pump holder 300 in which cannula 12 is inserted through an angled cannula port 304 that then pivots to be parallel with tray body 302, in this embodiment cannula port 504 is oriented at an angle to tray body 502 and remains at that angle for interfacing with pump. To enable this configuration, cannula port 504 includes a needle opening 508 (see FIG. 7C) that is separate from and oriented at an angle to the longitudinal opening 510 through which the cannula is inserted through the pump holder 500. Needle opening or side port 508 is positioned such that when the pump 400 is slid onto the pump holder 500 to releasably lock the pump 400 onto the holder 500, the transfer needle 402 of pump 400, which may be oriented generally parallel with tray body 502, slides through the needle opening 508 and into and through the septum 18 within the barrel 14 of the cannula 12 to establish fluid communication between the pump 400 and the cannula 12. In this manner, a fluidic path is established between the pump 400 and cannula 12 through a horizontally oriented transfer needle 402 and an angled delivery shaft 16 of the cannula.

Increasing the wear time of the disposable tray and cannula to seven days or more with the embodiments disclosed herein provides many advantages over current 3-day wear technology. Users are required to change out infusion sets less frequently which improves the user experience in several ways, including having to insert a needle into the body less often. Studies have further shown that angled insertions provide better and more consistent therapy than 90 degree infusion sets. The disposable parts also end up in the trash at least half as often, which reduces waste and provides improved environmental impact.

In embodiments, user-wearable infusion pump system can include a user-wearable infusion pump configured to contain a medicament and a pump holder configured to releasably hold the user-wearable infusion pump. The pump holder can include a base on which the user-wearable infusion pump is configured to retained. A cannula can be configured to be inserted through the pump holder and into a body of a user to deliver medicament from the user-wearable infusion pump into the body of the user. The cannula can be configured to be inserted into the body of the user at an angle between parallel and perpendicular to the base of the pump holder. The user-wearable infusion pump can be configured to be slid generally parallel along the base of the pump holder to releasably lock the user-wearable infusion pump onto the pump holder and interface the user-wearable infusion pump with the cannula.

In some embodiments, the pump holder includes a cannula port having a cannula opening configured to receive the cannula.

In some embodiments, the cannula opening is oriented at the angle between parallel and perpendicular to the base of the pump holder.

In some embodiments, the cannula port further includes a needle opening configured to receive a transfer needle of the user-wearable infusion pump therethrough to interface the user-wearable infusion pump with the cannula.

In some embodiments, the cannula comprises a septum configured to be pierced by the transfer needle and a cannula delivery shaft configured to be inserted into the body of the user at the angle between parallel and perpendicular to the base of the pump holder.

In some embodiments, the transfer needle is configured to pierce the septum at a different angle than the angle between parallel and perpendicular.

In some embodiments, the cannula port is configured to pivot from a first orientation with the cannula opening at the angle between parallel and perpendicular to the base of the pump holder following insertion of the cannula into the cannula port to a second orientation with the cannula opening parallel with the base of the pump holder.

In some embodiments, the user-wearable infusion pump is configured to be interfaced with the cannula when the cannula port is in the second orientation.

In some embodiments, a transfer needle of the user-wearable infusion pump is inserted through the cannula opening.

In some embodiments, in both the first orientation and the second orientation a cannula delivery shaft inserted into the body of the user is oriented at the angle between parallel and perpendicular.

In some embodiments, the pump holder further comprises a cannula shaft slot configured to orient a delivery shaft of the cannula extending into the body of the user at the angle between parallel and perpendicular in both the first orientation and the second orientation.

In embodiments, a pump holder for user-wearable infusion pump system is configured to releasably retain a user-wearable infusion pump on a body of a user. The pump holder can include a base on which the user-wearable infusion pump is configured to be retained and a cannula port including a cannula opening configured to receive a cannula inserted into the body of the user. The cannula opening can be oriented at an angle between parallel and perpendicular to the base. The user-wearable infusion pump can be configured to be slid generally parallel along the base to releasably lock the user-wearable infusion pump onto the pump holder and interface the user-wearable infusion pump with the cannula.

In some embodiments, the cannula port further includes a needle opening configured to receive a transfer needle of the user-wearable infusion pump therethrough to interface the user-wearable infusion pump with the cannula.

In some embodiments, the needle opening provides access to a septum of the cannula for piercing by the transfer needle.

In some embodiments, the cannula port is configured to enable the transfer needle to pierce the septum at an angle generally parallel to the base with the cannula opening oriented at the angle between parallel and perpendicular to the base.

In some embodiments, the cannula port is configured to pivot from a first orientation with the cannula opening at the angle between parallel and perpendicular to the base following insertion of the cannula into the cannula port to a second orientation with the cannula opening parallel with the base.

In some embodiments, the cannula port is configured to interface with the user-wearable infusion pump when the cannula port is in the second orientation.

In some embodiments, the cannula opening is configured to receive a transfer needle of the user-wearable infusion pump.

In some embodiments, the pump holder further includes a cannula shaft slot configured to orient a delivery shaft of the cannula at the angle between parallel and perpendicular in both the first orientation and the second orientation.

In some embodiments, the pump holder further includes one or more retention walls extending upwardly from a perimeter of the base, wherein the cannula port is monolithically formed with one of the one or more retention walls.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924 and commonly owned U.S. patent application Ser. Nos. 17/368,968; 17/896,492; 18/207,094; 18/398,543; 18/441,735; 18/474,839; 18/475,916; 18/478,552; 18/678,130; 18/700,168; 18/891,482; 18/896,045; and Ser. No. 18/962,169.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A user-wearable infusion pump system, comprising:
a user-wearable infusion pump configured to contain a medicament;
a pump holder configured to releasably hold the user-wearable infusion pump, the pump holder including a base on which the user-wearable infusion pump is configured to retained; and
a cannula configured to be inserted through the pump holder and into a body of a user to deliver medicament from the user-wearable infusion pump into the body of the user, wherein the cannula is configured to be inserted into the body of the user at an angle between parallel and perpendicular to the base of the pump holder, and
wherein the user-wearable infusion pump is configured to be slid generally parallel along the base of the pump holder to releasably lock the user-wearable infusion pump onto the pump holder and interface the user-wearable infusion pump with the cannula.

2. The user-wearable infusion pump system of claim 1, wherein the pump holder includes a cannula port having a cannula opening configured to receive the cannula.

3. The user-wearable infusion pump system of claim 2, wherein the cannula opening is oriented at the angle between parallel and perpendicular to the base of the pump holder.

4. The user-wearable infusion pump system of claim 3, wherein the cannula port further includes a needle opening configured to receive a transfer needle of the user-wearable infusion pump therethrough to interface the user-wearable infusion pump with the cannula.

5. The user-wearable infusion pump system of claim 4, wherein the cannula comprises a septum configured to be pierced by the transfer needle and a cannula delivery shaft configured to be inserted into the body of the user at the angle between parallel and perpendicular to the base of the pump holder.

6. The user-wearable infusion pump system of claim 5, wherein the transfer needle is configured to pierce the septum at a different angle than the angle between parallel and perpendicular.

7. The user-wearable infusion pump system of claim 2, wherein the cannula port is configured to pivot from a first orientation with the cannula opening at the angle between parallel and perpendicular to the base of the pump holder following insertion of the cannula into the cannula port to a second orientation with the cannula opening parallel with the base of the pump holder.

8. The user-wearable infusion pump system of claim 7, wherein the user-wearable infusion pump is configured to be interfaced with the cannula when the cannula port is in the second orientation.

9. The user-wearable infusion pump system of claim 8, wherein a transfer needle of the user-wearable infusion pump is inserted through the cannula opening.

10. The user-wearable infusion pump system of claim 7, wherein in both the first orientation and the second orientation a cannula delivery shaft inserted into the body of the user is oriented at the angle between parallel and perpendicular.

11. The user-wearable infusion pump system of claim 7, wherein the pump holder further comprises a cannula shaft slot configured to orient a delivery shaft of the cannula extending into the body of the user at the angle between parallel and perpendicular in both the first orientation and the second orientation.

12. A pump holder for user-wearable infusion pump system configured to releasably retain a user-wearable infusion pump on a body of a user, comprising:
a base on which the user-wearable infusion pump is configured to be retained; and
a cannula port including a cannula opening configured to receive a cannula inserted into the body of the user, the cannula opening being oriented at an angle between parallel and perpendicular to the base,
wherein the user-wearable infusion pump is configured to be slid generally parallel along the base to releasably lock the user-wearable infusion pump onto the pump holder and interface the user-wearable infusion pump with the cannula.

13. The pump holder of claim 12, wherein the cannula port further includes a needle opening configured to receive a transfer needle of the user-wearable infusion pump therethrough to interface the user-wearable infusion pump with the cannula.

14. The pump holder of claim 13, wherein the needle opening provides access to a septum of the cannula for piercing by the transfer needle.

15. The pump holder of claim 14, wherein the cannula port is configured to enable the transfer needle to pierce the septum at an angle generally parallel to the base with the cannula opening oriented at the angle between parallel and perpendicular to the base.

16. The pump holder of claim 12, wherein the cannula port is configured to pivot from a first orientation with the cannula opening at the angle between parallel and perpendicular to the base following insertion of the cannula into the cannula port to a second orientation with the cannula opening parallel with the base.

17. The pump holder of claim 16, wherein the cannula port is configured to interface with the user-wearable infusion pump when the cannula port is in the second orientation.

18. The pump holder of claim 17, wherein the cannula opening is configured to receive a transfer needle of the user-wearable infusion pump.

19. The pump holder of claim 16, further comprising a cannula shaft slot, the cannula shaft slot configured to orient a delivery shaft of the cannula at the angle between parallel and perpendicular in both the first orientation and the second orientation.

20. The pump holder of claim 12, further comprising one or more retention walls extending upwardly from a perimeter of the base, wherein the cannula port is monolithically formed with one of the one or more retention walls.
